(19) United States
(12) Patent Application Publication
(10) Pub. No.: US 2024/0374894 A1
(43) Pub. Date: Nov. 14, 2024

(54) COSMETIC DEVICE

(71) Applicant: NICORIO CO., LTD., Tokyo (JP)

(72) Inventor: Mitsunori SHISHIDA, Tokyo (JP)

(21) Appl. No.: 18/779,863

(22) Filed: Jul. 22, 2024

Related U.S. Application Data
(63) Continuation of application No. PCT/JP2022/039947, filed on Oct. 26, 2022.

(30) Foreign Application Priority Data
Jan. 26, 2022 (JP) 2022-010192

(51) Int. Cl. A61N 1/32; A61N 1/04; A61N 1/36; A61N 5/06

(52) U.S. Cl. CPC A61N 1/328; A61N 1/0452; A61N 1/36031; A61N 5/0616; A61N 2005/0626; A61N 2005/0651; A61N 2005/0663

(57) ABSTRACT

A cosmetic device includes a head, a grip, a treatment selection part, and a display. The grip is attached such that its distal end is coupled to the head body, with the head and grip being T-shaped as a whole. The head includes a first distal end and a second distal end. The first distal end is applied to a skin of a user, and includes a skin condition measuring part that measures the skin condition of the user. The second distal end is applied to the skin of the user, and includes an action part that electrically or mechanically acts on the skin. The treatment selection part diagnoses the measured skin condition and selects a corresponding treatment program. The display displays the selected treatment. The treatment selection part and the display are each provided at either one of the head and the grip.

### CROSS REFERENCE TO RELATED APPLICATIONS

This application is a Continuation of the U.S. National Stage of International Application No. PCT/JP2022/039947 filed on Oct. 26, 2022, which claims priority to Japanese Patent Application No. 2022-010192 on filed Jan. 26, 2022 under 35 U.S.C. § 119, the entire contents of all of which are hereby incorporated by reference.

### BACKGROUND

### 1. Technical Field

The present disclosure relates to a cosmetic device that acts on human skin.

### 2. Description of the Related Art

Conventionally, electronic cosmetic devices that perform various types of treatment according to beauty methods having different purposes and effects are known. For example, the EMS method is a method of electrically stimulating muscle to contract the muscle, and is effective in eliminating wrinkles. In the ion introduction method, an active ingredient contained in lotion or serum is ionized with weak current and permeated into the skin, thereby enhancing the function of the lotion or the serum.

In addition, there are beauty methods such as a method of promoting blood circulation and metabolism of the skin by light irradiation of an LED, a method of performing massage by vibration, a method of removing dirt clogged in old keratin or pores of the skin using fine vibration of ultrasonic waves, a method of activating cells by electrical stimulation by microcurrent (weak current), a method of warming the skin from a deep portion by radio waves, a method of tightening the skin by cooling, and a method of deriving ions to remove dirt of the skin, and electronic cosmetic devices corresponding thereto are used. In these treatment methods, it is desirable to select an optimal treatment method depending on the skin condition of a user.

Therefore, a technique in which a device for diagnosing the skin condition of the user is combined with the electronic cosmetic device as described above is known (see, for example, JP 2007-313149 A and JP 2004-141259 A).

The present discloser has recognized the following problems in realizing the techniques of JP 2007-313149 A and JP 2004-141259 A. In the technique of JP 2007-313149 A, since only one combination of treatment methods is provided, it is not possible to change the treatment method or select an optimal treatment method depending on the result of the skin condition. A user wants to purchase and use an electronic cosmetic device according to his/her skin affliction, but if the user purchases a plurality of devices, the cost increases accordingly. Since the skin condition changes, the same treatment is not always effective. A cosmetic device capable of performing various types of treatments is further desired. A cosmetic device capable of performing skin diagnosis and selecting a treatment program corresponding to the skin diagnosis is further desired.

In the technique of JP 2004-141259 A, a diagnostic device for diagnosing the skin condition (a controller main body or a computer in which a skin diagnostic application is installed) and a device for performing skin treatment are separately required. A cosmetic device in which the device for diagnosis and the device for treatment are integrated and not bulky and easy to use is further desired.

### SUMMARY

The present disclosure has been made in view of such problems, and an object of the present disclosure is to provide a cosmetic device with high operability capable of measuring a skin condition and selecting a treatment program corresponding to the skin condition.

In order to solve the above problems, the cosmetic device of the present disclosure includes: a head; a grip attached such that a distal end of the grip is joined to a body of the head; a treatment selection part structured to diagnose a measured skin condition and select a treatment program corresponding thereto; and a display structured to display a selected treatment, wherein the head and the grip are T-shaped as a whole, the head comprises a first distal end at an end in a longitudinal direction and a second distal end at an end facing the first distal end, the first distal end is applied to a skin of a user and comprises a skin condition measuring part that measures the skin condition of the user, the second distal end is applied to the skin of the user and comprises an action part that electrically or mechanically acts on the skin according to the treatment program, and the treatment selection part and the display are provided at either one of the head and the grip.

### BRIEF DESCRIPTION OF THE DRAWINGS

FIG. 1 is a front view of a cosmetic device according to Example 1;

FIG. 2 is a perspective view of the cosmetic device according to Example 1;

FIG. 3 is a view illustrating a skin condition measuring part of the cosmetic device according to Example 1;

FIG. 4 is a side cross-sectional view schematically illustrating the skin condition measuring part of the cosmetic device according to Example 1;

FIG. 5 is a block diagram illustrating control of treatment selection of the cosmetic device according to Example 1;

FIG. 6A and FIG. 6B are diagrams illustrating an example of a correspondence relationship between treatment program names and treatment contents, in which FIG. 6A is a table illustrating the treatment program names and functions thereof, and FIG. 6B is a table illustrating the treatment program names and action items thereof;

FIG. 7 is a view illustrating a display and a program selection button of the cosmetic device according to Example 1;

FIG. 8 is a diagram illustrating a use state of the cosmetic device according to Example 1;

FIG. 9 is a diagram illustrating a use state of the cosmetic device according to Example 1;

FIG. 10 is a view showing a state where a cosmetic device according to Example 2 stands on a charging stand; and

FIG. 11 is a view of a state in which cotton of the cosmetic device according to Example 2 is attached.

### DETAILED DESCRIPTION

The invention will now be described by reference to the preferred embodiments. This does not intend to limit the scope of the present invention, but to exemplify the invention.

Hereinafter, in the drawings, the same or equivalent components and members are denoted by the same reference numerals, and redundant description will be omitted as appropriate.

### EXAMPLE 1

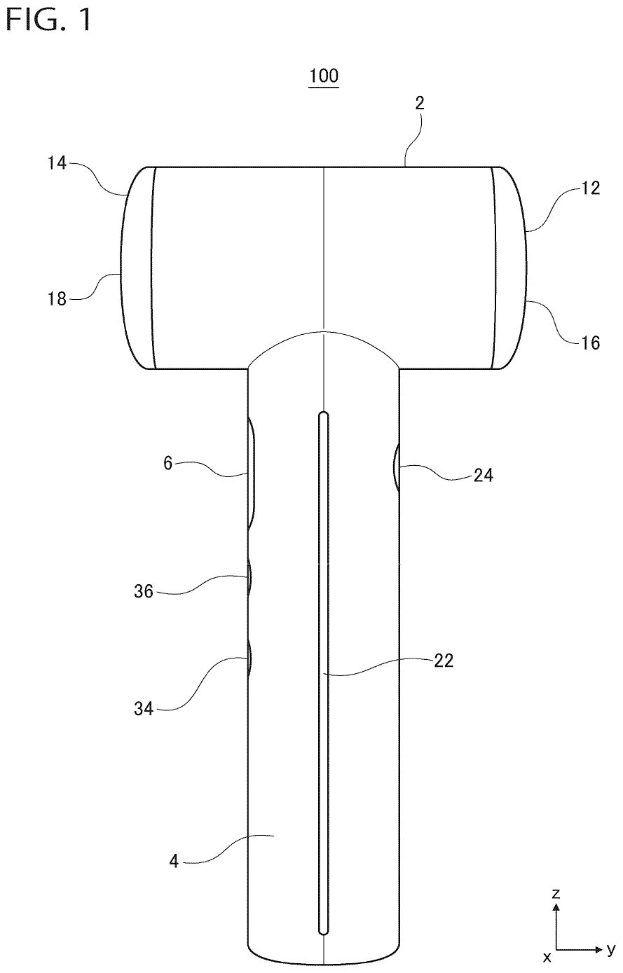

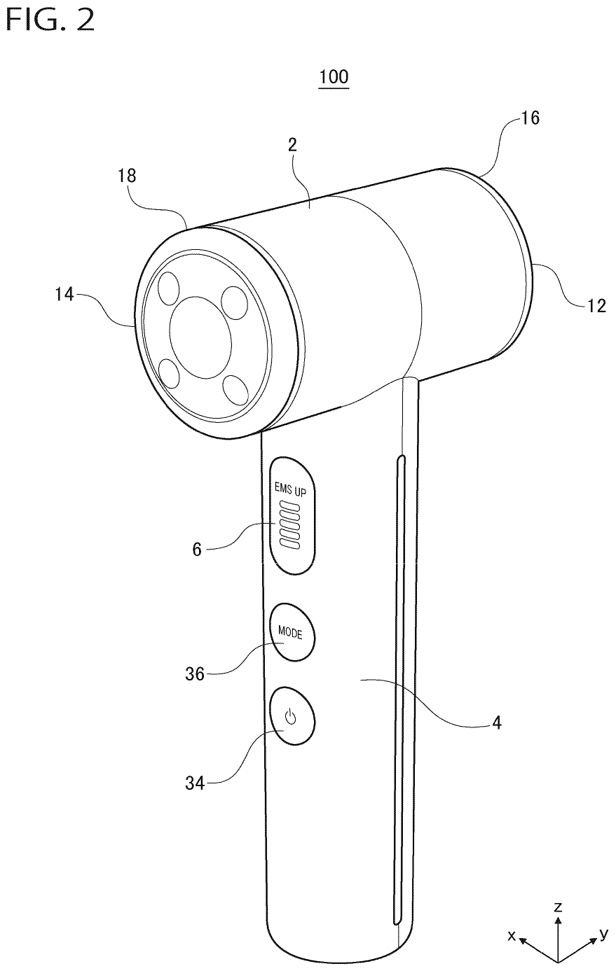

Hereinafter, a cosmetic device 100 of Example 1 will be described. FIG. 1 is a front view of the cosmetic device 100 according to Example 1. FIG. 2 is a perspective view of the cosmetic device according to Example 1. The cosmetic device 100 includes a head 2 and a grip 4. For convenience of description, as illustrated in the drawing, an xyz orthogonal coordinate system is defined in which a certain horizontal direction is an x direction, a horizontal direction orthogonal to the x direction is a y direction, and a direction orthogonal to both directions, that is, a vertical direction is a z direction. Such indication of a direction does not limit a use posture of the cosmetic device 100, and the cosmetic device 100 is used in an arbitrary posture according to an application.

The head 2 is formed in a cylindrical shape whose longitudinal direction is the x direction. The head 2 has a first distal end 16 and a second distal end 18. The first distal end 16 is applied to the skin of the user, and includes a skin condition measuring part 12 that measures the skin condition of the user. The second distal end 18 is applied to the skin of the user, and includes an action part 14 that electrically or mechanically acts on the skin. As a result of testing usability through experiments, the length of the head in the longitudinal direction is desirably 75 mm to 90 mm. The diameter of the cross section of the head is desirably 35 mm to 50 mm. The numerical range described below is also a range in which usability is confirmed by experiments.

The grip 4 is formed in a cylindrical shape whose longitudinal direction is the z direction. The grip 4 is integrally attached such that one distal end thereof is joined to the body of the head 2. A shutter switch 24 is provided on a side surface of the grip 4 toward which the first distal end 16 is directed. A display 6, a program selection button 36, and a power button 34 are provided on the side surface of the grip 4 toward which the second distal end 18 is directed. A contact 22 connected to the living body site using a member made of a conductive material is provided at a position where the contact is in contact with the skin when the user of the grip 4 grips the contact. The total height of the head 2 and the grip 4 in the z direction is desirably 150 mm to 180 mm.

The action part 14 is provided at the second distal end 18 which is one distal end of the head 2. The distal end surface of the action part 14 in contact with a skin of a user is covered with a transmissive member. In the distal end surface, at least a part of the edge part may be formed of a material having translucency. The action part 14 electrically or mechanically acts on the skin of the user brought into contact with the distal end face. Specifically, the action part 14 performs a treatment on the skin by any method or a combination of any method of EMS, ion derivation, ion introduction, light irradiation of blue LED, light irradiation of red LED, vibration, ultrasonic wave, microcurrent, radio wave (RF), and cooling. Note that the operation principle of a treatment such as EMS is known, and details are omitted.

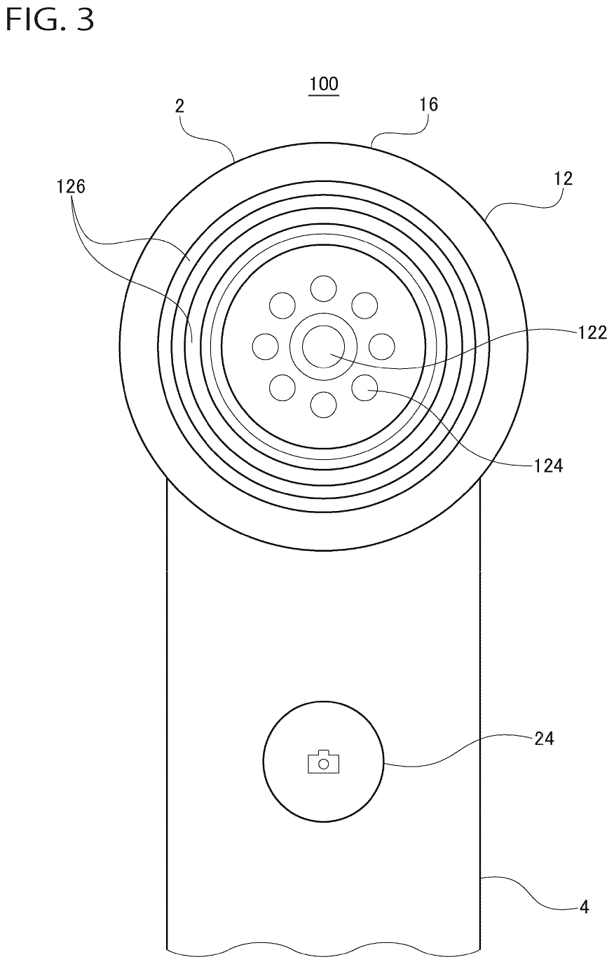

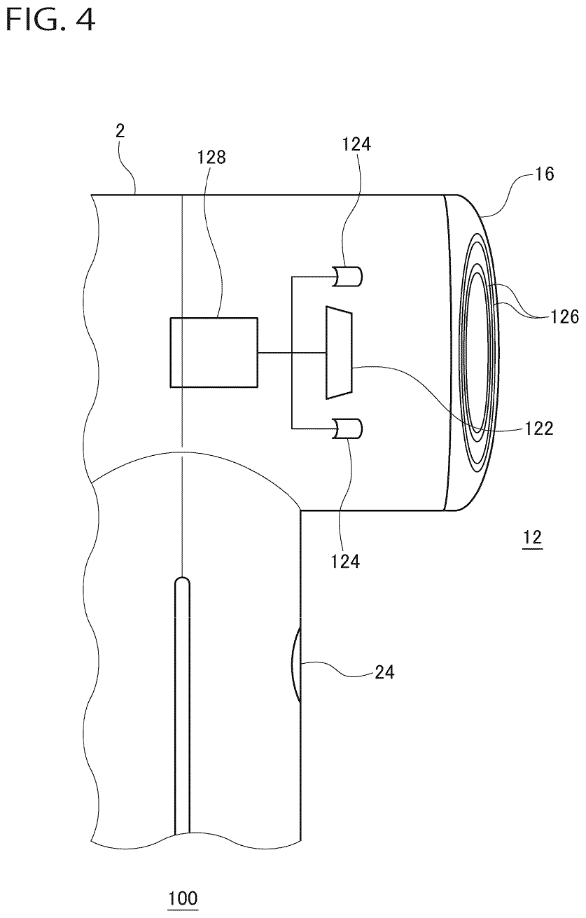

FIG. 3 is a view showing the skin condition measuring part 12 of the cosmetic device 100 according to Example 1, and FIG. 4 is a side sectional view schematically showing the skin condition measuring part 12 of the cosmetic device according to Example 1. The skin condition measuring part 12 includes a camera 122, a light irradiation 124, and a moisture sensor 126.

The moisture sensor 126 is provided on the distal end face of the first distal end 16. The moisture sensor 126 includes two annular electrodes having different diameters. The moisture sensor 126 measures the moisture amount of the skin of the user.

The camera 122 is provided inside the cylinder of the head 2. A central portion of a distal end face of the first distal end 16 of the head 2 is opened in a circular shape. The camera 122 is provided through the opening so as to be able to photograph the skin of the user brought into contact with the distal end face. A transparent cover material may be interposed between the camera 122 and the distal end face. As the light irradiation 124, a plurality of LEDs are arranged at equal distances along the periphery of the camera. The camera 122 and the light irradiation 124 are connected to a photographing control 128 provided at any place of the head 2 and the grip 4. The photographing control 128 controls photographing by the camera 122 and light irradiation by the light irradiation 124. The light irradiation 124 emits light in accordance with an instruction from the photographing control 128. A photosensor may be separately provided, and the light to be emitted may be adjusted according to the darkness.

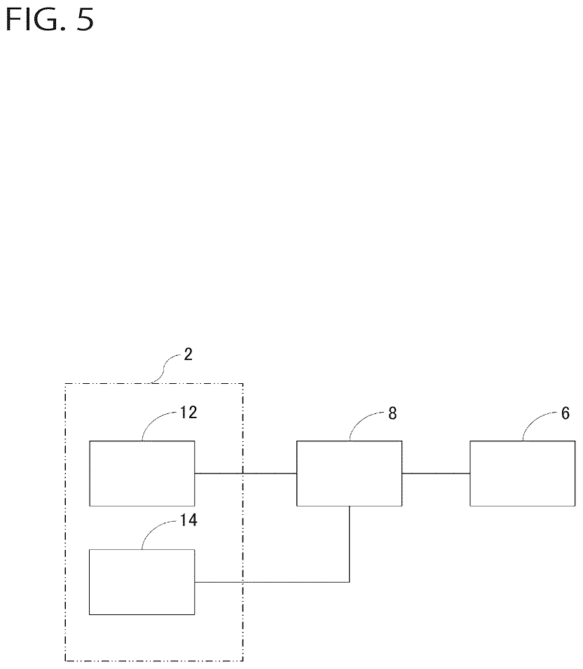

FIG. 5 is a block diagram illustrating control of treatment selection of the cosmetic device according to Example 1. The skin condition measuring part 12 is connected to a treatment selection part 8 that receives a result of the skin condition measurement. The treatment selection part 8 is connected to the display 6 that displays information such as the selected treatment program. The treatment selection part 8 is connected to the action part 14 that receives the selected treatment program.

The treatment selection part 8 acquires an image captured by the camera 122 of the skin condition measuring part 12, and evaluates texture, spots, blackheads, pores, and whitening degree. The treatment selection part 8 acquires skin moisture amount information measured by the moisture sensor 126 of the skin condition measuring part 12. The treatment selection part 8 selects an appropriate treatment program from the evaluation of the above image and the moisture amount information.

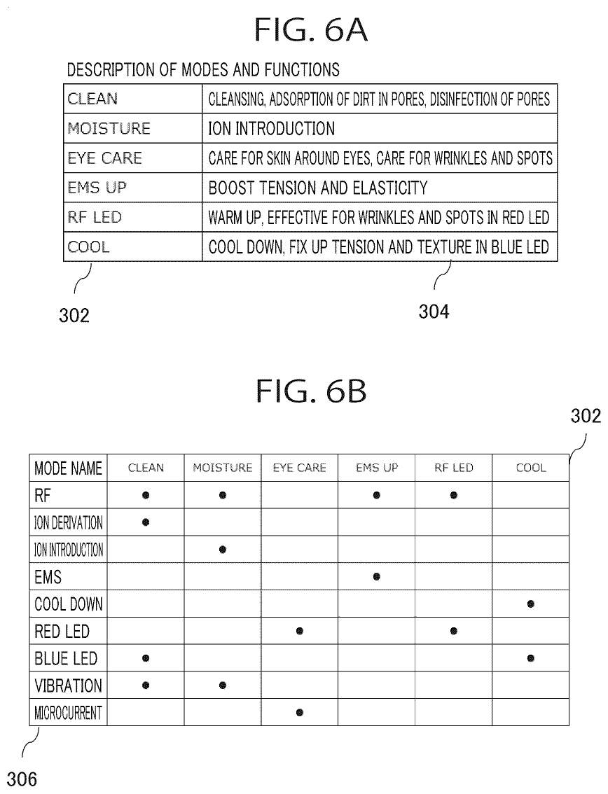

FIG. 6A and FIG. 6B are diagrams illustrating an example of a correspondence relationship between treatment program names 302 and treatment contents, in which FIG. 6A is a correspondence table illustrating the treatment program names 302 and treatment program functions 304, and FIG. 6B is a correspondence table illustrating the treatment program names 302 and action items 306. For example, in FIG. 6B, since the radio wave (RF) of the action item 306 and the EMS are selected in the “EMS UP” mode, a combination of these treatments is performed. In addition to the action item, the treatment program may include a combination of each action time and action intensity. The treatment program described herein is merely an example, and several tens or more kinds of treatment programs other than these treatment programs can be performed by combination.

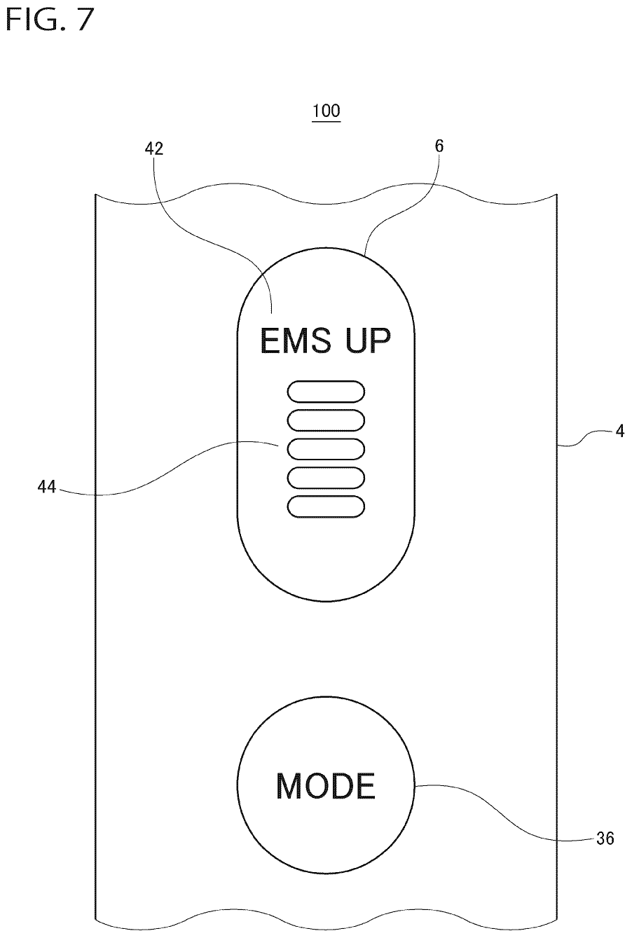

FIG. 7 is a view illustrating the display 6 and the program selection button 36 of the cosmetic device 100 according to Example 1. A treatment selection part 8 sends the selected treatment program name to the display 6, and the display 6 displays the treatment program name on a treatment program display 42. An indicator 44 displays the intensity level of the treatment. The indicator 44 may display the remaining time of the treatment. The display 6 may change the color to be displayed according to the treatment program. With the above configuration, the cosmetic device 100 is used as follows.

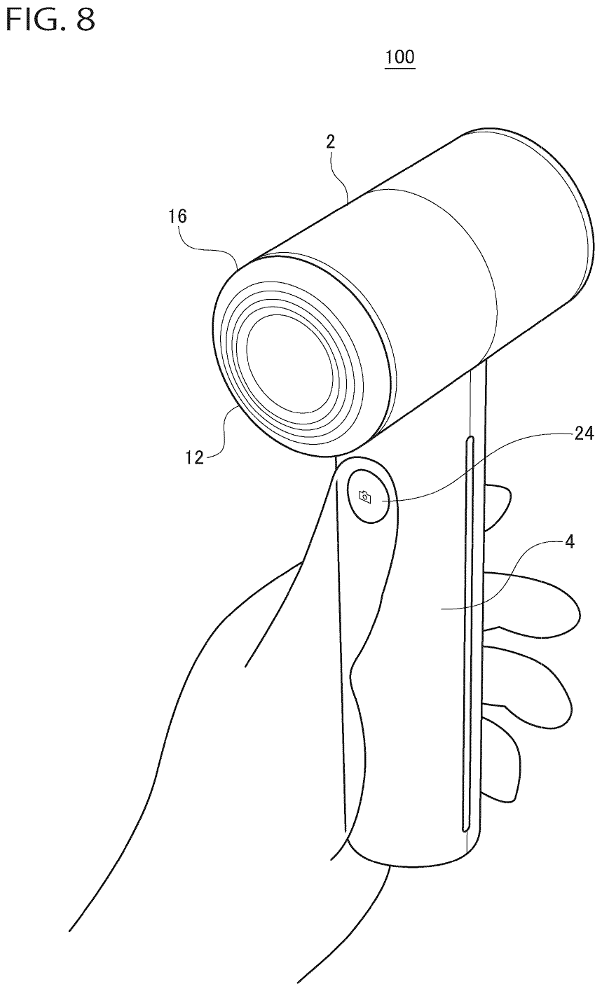

FIG. 8 is a view illustrating a use state of the cosmetic device according to Example 1. The user faces the first distal end 16 toward the user, and grips the first distal end 16 at a position where the thumb of the user touches the shutter switch 24. The user brings the skin condition measuring part 12 into close contact with his/her skin and presses the shutter switch 24. The photographing control 128 of the skin condition measuring part 12 causes the light irradiation 124 to irradiate the skin of the user with light, and causes the camera 122 to photograph the skin of the user. The skin condition measuring part 12 sends the photographed image data to the treatment selection part 8. The moisture sensor 126 of the skin condition measuring part 12 measures the moisture amount of the skin of the user. The skin condition measuring part 12 sends the measurement information to the treatment selection part 8.

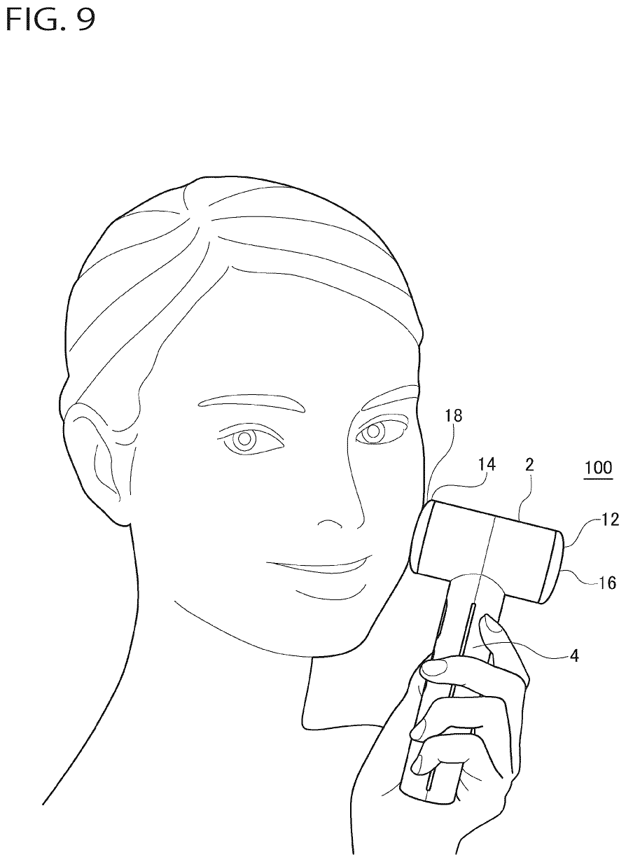

FIG. 9 is a view illustrating a use state of the cosmetic device according to Example 1. The user rotates the grip 4 by 180 degrees about the z direction, and turns the second distal end 18 toward the user. The treatment selection part 8 selects an appropriate treatment program from the acquired image data and moisture amount information.

The treatment selection part 8 sends the selected treatment program name 302 to the display 6. The display 6 displays the treatment program display 42. The treatment selection part 8 sends the selected treatment program to the action part 14.

The user checks the treatment program display 42 displayed on the display 6 and presses the action part 14 against his/her skin. The action part 14 executes a treatment according to the selected treatment program. The user can switch to another program by pressing the program selection button 36.

The cosmetic device 100 has a secondary battery (not shown) in a main body. As in Example 2 described later, charging may be performed from a dedicated charger, or a charging port (not shown) for charging from an external adapter (not shown) may be provided. The secondary battery has a battery capacity of 2000 mAh, and can be operated for 40 minutes by charging for 2.5 hours.

With the above configuration, the following effects are obtained.

The user can automatically select an optimal treatment program suitable for his/her skin. The user can diagnose his/her skin with a simple operation, and can execute a treatment program corresponding to the diagnosis result. For example, the user can switch between skin diagnosis and treatment only by rotating the cosmetic device 100 by 180 degrees in the hand, and can execute a series of actions with seamless movement. The user can execute various types of treatment programs with one device.

Since the distal end surface of the action part 14 in contact with the skin of the user is covered with a member having translucency, the user can visually recognize the light irradiation of the blue LED and the light irradiation of the red LED.

### EXAMPLE 2

Hereinafter, a cosmetic device 100 of Example 2 will be described.

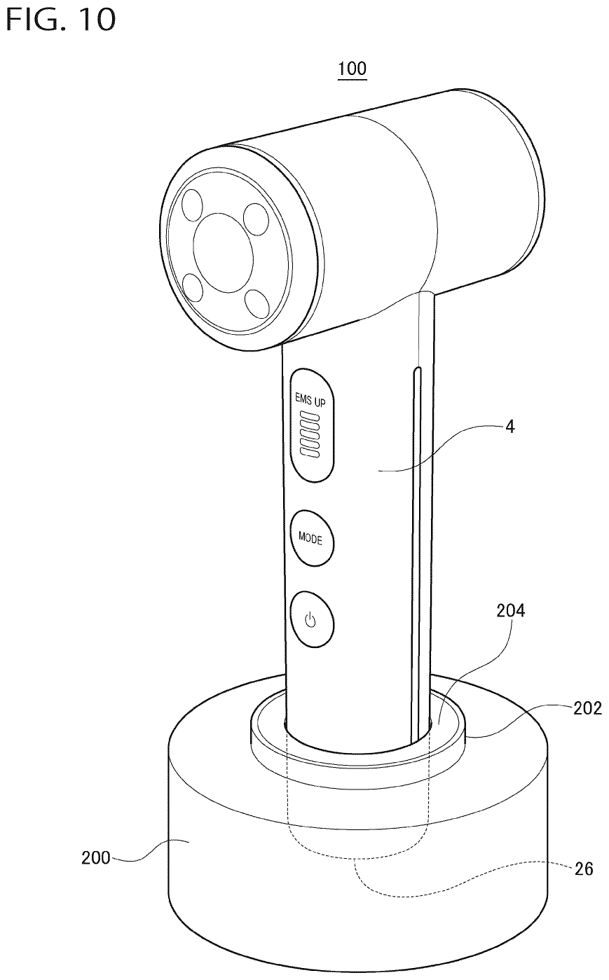

FIG. 10 is a view of the cosmetic device 100 according to Example 2 standing on a charging stand 200. A grip 4 has a charge receiving part 26 at an end farther from a joined head 2. The charging stand 200 enables contactless charging. The charging stand 200 has, at the center of its upper surface, a recess into which an end of the grip 4 of the cosmetic device 100 can be inserted and placed upright. When the cosmetic device 100 is placed upright on the charging stand 200, electricity is supplied from the charging stand 200 through the charge receiving part 26.

The charging stand 200 has a cotton-attached ring stopper 204 on which a cotton-attached ring 202 can be placed at the center of the upper surface. The cotton-attached ring 202 is annular and has a diameter larger than the diameter of the cross section of the head by the thickness of cotton to be described later.

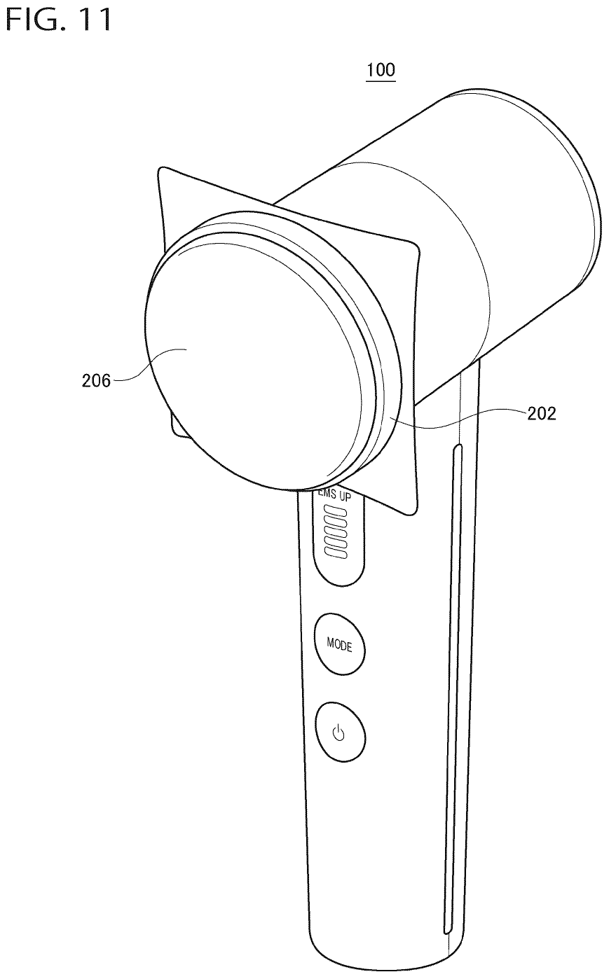

FIG. 11 is a view of a state in which a cotton 206 of the cosmetic device 100 according to Example 2 is attached. The user applies the cotton 206 to an action part 14, and fixes the cotton 206 with the cotton-attached ring 202. In a case where the treatment of ion derivation is selected or in a case where the treatment of ion derivation is selected and executed, when the treatment is executed by the action part 14, dirt in pores and a makeup residue that cannot be removed by face washing or cleansing are floated and adsorbed to the cotton by the force of ions.

With the above configuration, the following effects are obtained.

The charging stand 200 of Example 2 can charge the cosmetic device 100 while storing cosmetic device 100. Even when charging is unnecessary, the cosmetic device 100 can be stored in a beautiful and compact manner. Since the cotton 206 can be disposable, it is hygienic. The action part 14 can be kept clean. The risk of losing the cotton-attached ring 202 is reduced.

The embodiments of the underlying technology have been described above. It is to be understood by those skilled in the art that the exemplary embodiment is an example, various modifications are possible, and such modifications are also included in the underlying technology.

### Modification 1

In Example 1, the shutter switch 24, which is a button for operating the skin condition measuring part 12, is provided on the side toward which the first distal end 16 of the grip 4 is directed, but the shutter switch 24 may be provided on one of the side surfaces of the grip 4 on the side toward which the direction (x direction) orthogonal to the direction (y direction) is directed. In this case, the program selection button 36, which is a button for operating the action part 14, is provided on the side surface of the grip 4 on the side facing the shutter switch 24 across the axis of the grip 4.

In Example 1, operability is high for the user to operate with the thumb, but in the present modification, operability is high for the user to operate with the index finger.

### Modification 2

In Example 1, the distal end surface of the action part 14 in contact with the skin of the user is covered with a member having translucency, but only the entire circumference of the edge part of the distal end may be formed of a material having translucency. As in Example 1, the user can visually recognize the light irradiation of the blue LED and the light irradiation of the red LED.

### Modification 3

In Example 1, the distal end surface of the action part 14 in contact with the skin of the user is covered with a member having translucency, but a part of the edge part of the distal end may be formed of a material having translucency. As in Example 1, the user can visually recognize the light irradiation of the blue LED and the light irradiation of the red LED.

### Modification 4

Although the display 6 indicating the treatment being executed is provided on the side toward which the second distal end 18 is directed, the display 6 may be provided on one of the side surfaces of the grip 4 on the side toward which the direction (x direction) orthogonal to the direction (y direction) is directed.

In use, the user can visually recognize the treatment program display 42 by viewing the display 6 reflected in the mirror on the side surface.

### Modification 5

The cosmetic device 100 may realize communication by a known wireless communication technology or a wired communication technology, and display result data measured by the skin condition measuring part 12 on a terminal such as a smartphone.

### Modification 6

In Example 1, the moisture amount of the skin is measured by the moisture sensor 126, but an oil sensor may be provided instead of the moisture sensor to measure the oil amount of the skin, or an oil sensor may be provided together with the moisture sensor 126. More data could enable more complex diagnoses of skin conditions.

### Modification 7

In Example 1, the distal end surface of the action part 14 in contact with the skin of the user is covered with the transmissive member, but a spacer may be provided so that the light irradiation of the blue LED and the light irradiation of the red LED leaking therefrom can be visually recognized.

What is claimed is:

1. A cosmetic device comprising:
a head;
a grip attached such that a distal end of the grip is joined to a body of the head;
a treatment selection part structured to diagnose a measured skin condition and select a treatment program corresponding thereto; and
a display structured to display a selected treatment,
wherein the head and the grip are T-shaped as a whole,
the head comprises a first distal end at an end in a longitudinal direction and a second distal end at an end facing the first distal end,
the first distal end is applied to a skin of a user and comprises a skin condition measuring part that measures the skin condition of the user,
the second distal end is applied to the skin of the user and comprises an action part that electrically or mechanically acts on the skin according to the treatment program, and
the treatment selection part and the display are each provided at either one of the head and the grip.
